(19) United States
(12) Patent Application Publication
(10) Pub. No.: US 2008/0050723 A1
(43) Pub. Date: Feb. 28, 2008

(54) Molecular method for diagnosis of colon cancer

(76) Inventors: Nabil Belacel, Moncton (CA); Miroslava Cuperlovic-Culf, Moncton (CA); Rodney Quelette, Dieppe (CA)

Correspondence Address: Shapiro Cohen, P.O. Box 13002, Kanata, ON, K2K 0E2, US

(21) Appl. No.: 11/508,244

(22) Filed: Aug. 23, 2006

(51) Int. Cl. C12Q 1/68

(52) U.S. Cl. 435/6

(57) ABSTRACT

Methods for diagnosing or detecting cancerous colon tissue. A panel of 21 specific marker genes are provided. The overexpression of some of these marker genes compared to their expression in normal colon tissue and the underexpression of the rest of these marker genes are indicative of cancerous colon tissue. By using these 21 marker genes as a diagnostic tool, smaller tissue samples, such as those obtained by core needle biopsies and from patient stool samples, can be used.

### FIELD OF THE INVENTION

The present invention relates to diagnosis methods and, more particularly, to diagnosis methods for detecting colon cancer.

### BACKGROUND TO THE INVENTION

With 19,200 new cases in Canada in 2004, colon cancer is one of the three most prevalent cancers in Canada for both men and women (Canadian Cancer Statistiscs, 2004). Invasive biopsy procedures require long hospitalizations and may have numerous possible side effects. Other alternative diagnostic procedures, such as digital rectal examination, fecal occult blood procedure, double-contrast barium enema, flexible sigmoidoscopy, and total colonoscopy are mostly invasive. The fecal occult blood test, while non-invasive, requires confirmation by way of additional invasive procedures. Unfortunately, such invasive procedures can possibly lead to side effects and/or long hospitalizations.

There is therefore a need for a non-invasive and accurate testing procedure for detecting colon cancer. Ideally, such a test should be able to detect cancerous colon cells even from small sample sizes.

There is therefore a need for a more accurate diagnostic method that does not require an invasive biopsy to detect or diagnose colon cancer. Ideally, such a method should be usable even with very small sample sizes and may be combined with other, pathologist-based diagnosis methods.

### SUMMARY OF THE INVENTION

The present invention provides methods for diagnosing or detecting cancerous colon tissue. Colon tissue samples are acquired and are tested for the expression of specific marker genes. A panel of 21 specific marker genes are provided. The overexpression of some of these marker genes compared to their expression in normal colon tissue and the underexpression of the rest of these marker genes compared to normal colon tissue are indicative of cancerous colon tissue. By using these 21 marker genes as a diagnostic tool, small tissue samples, such as those obtained by core needle biopsies and from stool samples can be used.

In a first aspect, the present invention provides a method for determining if colon cells are cancerous, the method comprising:

a) obtaining said colon cells;

b) determining if at least one specific gene is overexpressed or underexpressed in said colon cells compared to an expression of said at least one specific gene in normal colon cells;

c) determining that said colon cells are cancerous based on whether said at least one gene is overexpressed or underexpressed in said colon cells.

In another aspect, the present invention provides a use of at least one marker gene for identifying cancerous colon tissue, an overexpression or underexpression of said at least one marker gene in colon tissue compared to an expression of said at least one marker gene in normal colon tissue being indicative of cancerous colon tissue.

Yet another aspect of the invention provides a method of diagnosing colon cancer, the method comprising:

a) obtaining colon tissue to be diagnosed;

b) determining if specific marker genes are overexpressed or underexpressed in said colon tissue to be diagnosed compared to non-cancerous colon tissue;

c) determining if said colon tissue to be diagnosed is cancerous based on an underexpression or overexpression of said specific marker genes.

### BRIEF DESCRIPTION OF THE DRAWINGS

A better understanding of the invention will be obtained by considering the detailed description below, with reference to the following drawings in which:

FIG. 1 is an expression plot for the 21 genes which is the subject of the present invention;

FIGS. 2-20 illustrate box plots of the expression of the 21 genes in both cancerous and non-cancerous tissue; and

FIG. 21 is a table which, taken in conjunction with a table in the description, denotes which sample sets were used in which experiments for the box plotted results in FIGS. 2-20.

### DETAILED DESCRIPTION

The present invention relates to the use of a panel of 21 specific marker genes to diagnose or detect cancerous colon tissue. The panel of 21 marker genes is listed in Table 1 below. Experiments have shown that this panel of marker genes give high accuracy in colon cancer diagnosis due to the expression levels of the marker genes in cancer tissue relative to their expression levels in normal tissue.

The panel of 21 marker genes is given in Table 1. The marker genes were determined from two different microarray data sets. The first 14 genes were found to give 100% of correct classification for the data set described by Notterman D A, et al. ((2001) Transcriptional Gene Expression Profiles of Colorectal Adenoma, Adenocarcinoma and Normal Tissue Examined by Oligonucleotide Arrays. Cancer Res. 61:3124-3130). The rest of the genes in the panel were selected from the data set published by Alon, U. et al. ((1999) Broad Patterns of Gene Expression Revealed by Clustering Analysis of Tumour and Normal Colon Tissue Probed by Oligonucleotide Arrays. Proc. Natl. Acad. Sci. 96: 6745-6750).

The data set from Alon, et al. consisted of 40 tumour and 22 normal samples for a total of 66 samples. Samples were obtained from colon adenocarcinoma specimens snap-frozen in liquid nitrogen within 20 min of removal/collection from patients. From some of these patients paired normal colon tissue also was obtained. The microarrays were hybridized using Affymetrix Hum600 array using standard protocol. The 2,000 highest intensity genes were selected and published on the web at http://microarray.princeton.edu/oncology/. From this subset were selected seven diagnostic genes that give 100% of correct classification (the last 6 genes in Table 1). The dataset from Alon et al. is limited in size and therefore biomarker selection was performed on another data set also found in the Notterman et al. paper. In this data set, samples of colon adenocarcinoma and paired normal tissue from the same patient were obtained from the Cooperative Human Tissue Network. The tissue was snap-frozen in liquid nitrogen within 20-30 min of harvesting and stored thereafter at −80° C. mRNA was extracted from the bulk tissue samples and hybridized to the array using standard procedure (see Notterman et al., 2001). This data set was also cited by Rhodes et al. in 2004 (see Rhodes, D. R. et al. (2004) Large-scale Meta-Analysis of Cancer Microarray Data Identifies Common Transcriptional Profiles of Neoplastic Transformation and Progression. Proc. NatI. Acad. Sci. 101: 9309). The adenocarcinoma samples were specifically re-reviewed by a pathologist at the institution where the samples were obtained using paraffin-embedded tissue that was adjacent or in close proximity to the frozen sample from which the RNA was extracted. The publicly available data set consists of 18 adenocarcinoma and 18 normal samples. The set consists of ˜6600 genes. The 14 genes that give 100% accurate diagnosis of adenocarcinomas and normal colon tissue were selected using another method.

TABLE 1
Panel of 21 genes found to give high accuracy in colon
cancer diagnosis and their expression level in cancer
relative to normal tissue.

| | | | Over or Under- |
|---|---|---|---|
| | | | expressed |
| GeneBank | | | in cancer |
| Accession | | | tissue relative to |
| Number | Gene Name | Symbol | normal tissue |
| M77836 | Pyrroline-5-carboxylate | PYCR1 | Overexpressed |
| | reductase 1 | | |
| X63468 | General transcription | GTF2E1 | Over-expressed |
| | factor IIE, polypeptide 1, | | |
| | alpha 56 kDa | | |
| H20426 | Transcribed locus, | NME1 | Over-expressed |
| | strongly similar to | | |
| | NP_937818.1 nucleoside- | | |
| | diphosphate kinase 1 | | |
| | isoform a [Homo sapiens] | | |
| L18960 | Eukaryotic translation | EIF1AX | Over-expressed |
| | initiation factor 1A, X- | | |
| | linked | | |
| U30872 | Centromere protein F, | CENPF | Over-expressed |
| | 350/400ka (mitosin) | | |
| X81438 | Amphiphysin (Stiff-Man | AMPH | Over-expressed |
| | syndrome with breast | | |
| | cancer 128 kDa | | |
| | autoantigen) | | |
| H67367 | RAN binding protein 1 | RANBP1 | Over-expressed |
| D13645 | KIAA0020 | KIAA0020 | Over-expressed |
| R33367 | Membrane cofactor | MCP | Over-expressed |
| | protein (CD46, | | |
| | trophoblast-lymphocyte | | |
| | cross-reactive antigen) | | |
| T94834 | similar to Homo sapiens | similar to | Over-expressed |
| | acidic (leucine-rich) | ANP32B | |
| | nuclear phosphoprotein | | |
| | 32 family, member B. | | |
| L20852 | Solute carrier family 20 | SLC20A2 | Under-expressed |
| | (phosphate transporter), | | |
| | member 2 | | |
| R39540 | TU3A protein | TU3A | Under-expressed |
| H86039 | Adenylate kinase 1 | AK1 | Under-expressed |
| T78477 | Zinc finger protein 297 | ZNF297 | Under-expressed |
| T84082 | ER Lumen Protein | KDELR1 | Under-expressed |
| | Retaining Receptor 1 | | |
| X05610 | Human mRNA for type IV | COL4A2 | Under-expressed |
| | collagen alpha (2) chain | | |
| R39130 | S27965 Hypothetical | LOC51035 | Under-expressed |
| | protein | | |
| T49397 | Src homology 2 domain | SHC | Under-expressed |
| | contating transforming | | |
| | protein 1 | | |
| T94350 | Peripheral myelin protein | PMP22 | Under-expressed |
| | 22 | | |
| L42611 | Keratin 6 isoform K6e | KRT6E | Under-expressed |
| | mRNA | | |
| M33653 | Collagen, type XIII, | COL13A1 | Under-expressed |
| | alpha 1 | | |

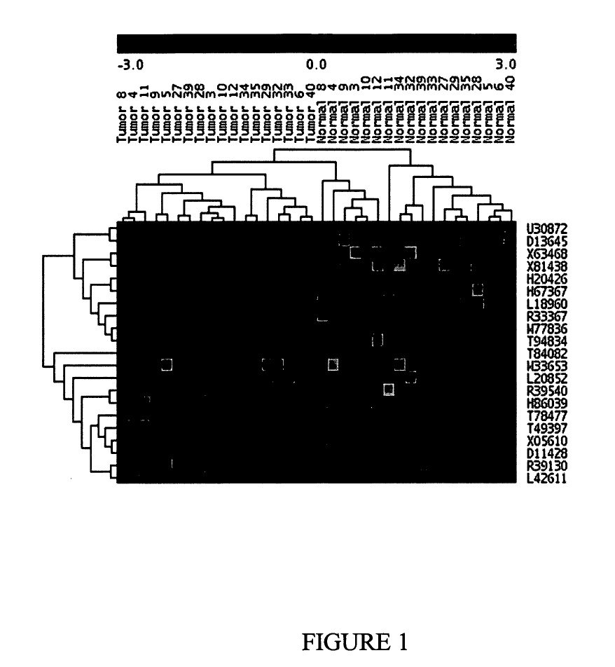

The genes listed above were derived using a microarray gene expression experiment, the gene expression plot being provided as FIG. 1 for the 21 genes. For this expression plot, the samples are normal and cancerous tissues. In the plot, positive expression levels are shown in red while negative expression levels are shown in green. All experiments were normalized (scaled) to have a mean of zero and a standard deviation of one.

By following the procedure noted above, the expression of the above genes can be determined from sample tissue obtained from a patient. By determining the expression of the above noted genes in the sample tissue, the presence or absence of cancerous colon tissue may be determined.

It should be noted that the procedure for determining the expression of genes in tissue is well-known in the art. Furthermore, procedures for the extraction and collection of tissue, in this case colon tissue, are also well-known. As noted above, colon tissue samples may be obtained from patient stool samples or core needle biopsies. These tissue samples may then be tested for the expression of the above genes and then compared to the expression of the above genes in tissue samples known to be non-cancerous. If the first 10 genes listed above are overexpressed in the patient sample tissue relative to their expression levels in normal tissue, and if the next 11 genes listed above are underexpressed in the patient sample tissue relative to their expression levels in normal tissue, then this would indicate the presence of cancerous colon tissue in the patient sample tissue.

It should be noted that expression analysis can be carried out using any method for measuring gene expression. Such methods as microarrays, diagnostic panel mini-chip, PCR, real-time PCR, and other similar methods may be used. Similarly, methods for measuring protein expression may also be used.

As noted above, the cancerous colon cells can be obtained from a patient using minimally invasive core needle biopsy or from techniques such as from a patient's stool samples. Normal or non-cancerous colon cells against which the cancerous cells can be compared can also be obtained from the patient or from other patients. Experiments have shown that the diagnosis can be possible from just a small number of cancer cells.

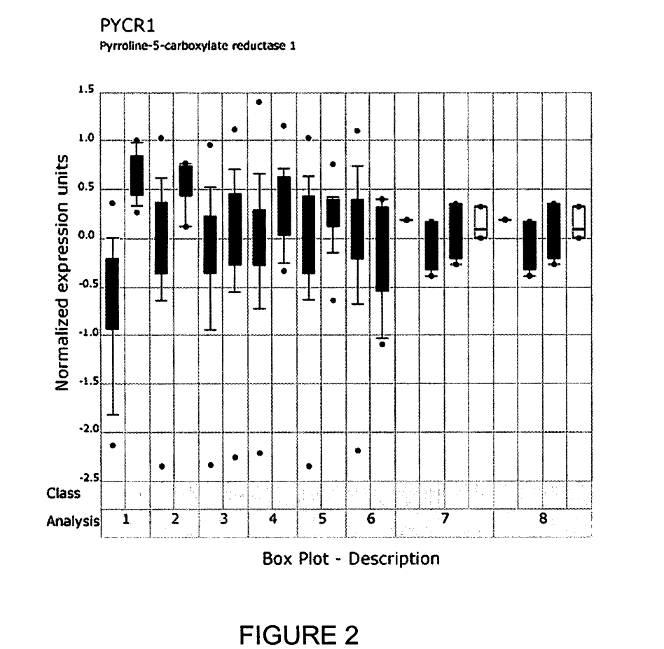

Referring to FIGS. 2-20, boxplots of test results for the above noted genes are illustrated. The boxplots illustrate that, for each particular gene, that gene is either underexpressed or overexpressed in cancerous tissue relative to normal tissue. The tissue samples which were used for the experiments were those used and referred to in the following publications as set out in the table below:

| Sample | | Sample | |
|---|---|---|---|
| Set | Publication | subset | Sample type |
| A | Notterman DA, Alon U, Sierk AJ, | 1 | Normal tissue |
| | Levine AJ. Transcriptional gene | 2 | Adenocarcionoma tissue |
| | expression profiles of colorectal | | |
| | adenoma, adenocarcinoma, and | | |
| | normal tissue examined by | | |
| | oligonucleotide arrays. Cancer | | |
| | Res. 2001 Apr 1; 61(7): 3124–30 | | |
| B | Zou TT, Selaru FM, Xu Y, | 1 | normal colonic |
| | Shustova V, Yin J, Mori Y, | | epithelium |
| | Shibata D, Sato F, Wang S, Olaru A, | 2 | colon carcinoma |
| | Deacu E, Liu TC, Abraham JM, | | |
| | Meltzer SJ. Application of cDNA | | |
| | microarrays to generate a | | |
| | molecular taxonomy capable of | | |
| | distinguishing between colon | | |
| | cancer and normal colon. | | |
| | Oncogene. 2002 Jul | | |
| | 18; 21(31): 4855–62. | | |
| C | Notterman DA, Alon U, Sierk AJ, | 1 | Duke Stage A |
| | Levine AJ. Transcriptional gene | 2 | Duke Stage B |
| | expression profiles of colorectal | 3 | Duke Stage C |
| | adenoma, adenocarcinoma, and | 4 | Duke Stage D |
| | normal tissue examined by | | |
| | oligonucleotide arrays. Cancer | | |
| | Res. 2001 Apr 1; 61(7): 3124–30 | | |
| D | Notterman DA, Alon U, Sierk AJ, | 1 | Stage A(1) |
| | Levine AJ. Transcriptional gene | 2 | Stage B(7) |
| | expression profiles of colorectal | 3 | Stage C(5) |
| | adenoma, adenocarcinoma, and | 4 | Stage D(5) |
| | normal tissue examined by | | |
| | oligonucleotide arrays. Cancer | | |
| | Res. 2001 Apr 1; 61(7): 3124–30. | | |
| E | Notterman DA, Alon U, Sierk AJ, | 1 | p53 mutation negative |
| | Levine AJ. Transcriptional gene | 2 | p53 mutation positive |
| | expression profiles of colorectal | | |
| | adenoma, adenocarcinoma, and | | |
| | normal tissue examined by | | |
| | oligonucleotide arrays. Cancer | | |
| | Res. 2001 Apr 1; 61(7): 3124–30. | | |
| F | Shyamsundar R, Kim YH, Higgins JP, | 1 | Multitissue |
| | Montgomery K, Jorden M, | 2 | Colon Normal |
| | Sethuraman A, van de Rijn M, | | |
| | Botstein D, Brown PO, Pollack JR. | | |
| | A DNA microarray survey of gene | | |
| | expression in normal human | | |
| | tissues. Genome Biol. | | |
| | 2005; 6(3): R22. Epub 2005 Feb 14 | | |
| G | Notterman DA, Alon U, Sierk AJ, | 1 | Female |
| | Levine AJ. Transcriptional gene | 2 | Male |
| | expression profiles of colorectal | | |
| | adenoma, adenocarcinoma, and | | |
| | normal tissue examined by | | |
| | oligonucleotide arrays. Cancer | | |
| | Res. 2001 Apr 1; 61(7): 3124–30. | | |
| H | Ramaswamy S, Tamayo P, Rifkin R, | 1 | Cancer progression normal |
| | Mukherjee S, Yeang CH, | | |
| | Angelo M, Ladd C, Reich M, | 2 | cancer progression primary |
| | Latulippe E, Mesirov JP, Poggio T, | | |
| | Gerald W, Loda M, Lander ES, | | |
| | Golub TR. Multiclass cancer | | |
| | diagnosis using tumor gene | | |
| | expression signatures. Proc Natl | | |
| | Acad Sci USA. 2001 Dec 18; 98 | | |
| I | Su AI, Welsh JB, Sapinoso LM, | 1 | multi-tissue cancer |
| | Kern SG, Dimitrov P, Lapp H, | 2 | colorectal |
| | Schultz PG, Powell SM, Moskaluk CA, | | adenocarcinoma |
| | Frierson HF Jr, Hampton GM. | | |
| | Molecular classification of human | | |
| | carcinomas by use of gene | | |
| | expression signatures. Cancer Res. | | |
| | 2001 Oct 15; 61(20): 7388–93. | | |
| J | Ramaswamy S, Tamayo P, Rifkin R, | 1 | Multi-tissue cancer |
| | Mukherjee S, Yeang CH, | 2 | colorectal |
| | Angelo M, Ladd C, Reich M, | | adenocarcinoma |
| | Latulippe E, Mesirov JP, Poggio T, | | |
| | Gerald W, Loda M, Lander ES, | | |
| | Golub TR. Multiclass cancer | | |
| | diagnosis using tumor gene | | |
| | expression signatures. Proc Natl | | |
| | Acad Sci USA. 2001 Dec 18; 98 | | |
| K | Ramaswamy S, Tamayo P, Rifkin R, | 1 | primary |
| | Mukherjee S, Yeang CH, | 2 | metastatic |
| | Angelo M, Ladd C, Reich M, | | |
| | Latulippe E, Mesirov JP, Poggio T, | | |
| | Gerald W, Loda M, Lander ES, | | |
| | Golub TR. Multiclass cancer | | |
| | diagnosis using tumor gene | | |
| | expression signatures. Proc Natl | | |
| | Acad Sci USA. 2001 Dec 18; 98 | | |
| L | Ramaswamy S, Tamayo P, Rifkin R, | 1 | Primary |
| | Mukherjee S, Yeang CH, | 2 | metastatic |
| | Angelo M, Ladd C, Reich M, | | |
| | Latulippe E, Mesirov JP, Poggio T, | | |
| | Gerald W, Loda M, Lander ES, | | |
| | Golub TR. Multiclass cancer | | |
| | diagnosis using tumor gene | | |
| | expression signatures. Proc Natl | | |
| | Acad Sci USA. 2001 Dec 18; 98 | | |
| M | Alon U, Barkai N, Notterman DA, | 1 | normal colon |
| | Gish K, Ybarra S, Mack D, Levine AJ. | 2 | colon adenocarcinoma |
| | Broad patterns of gene | | |
| | expression revealed by clustering | | |
| | analysis of tumor and normal colon | | |
| | tissues probed by oligonucleotide | | |
| | arrays. Proc Natl Acad Sci USA. | | |
| | 1999 Jun 8; 96 | | |
| N | Ramaswamy S, Tamayo P, Rifkin R, | 1 | multi-tissue normal |
| | Mukherjee S, Yeang CH, | 2 | Colon normal |
| | Angelo M, Ladd C, Reich M, | | |
| | Latulippe E, Mesirov JP, Poggio T, | | |
| | Gerald W, Loda M, Lander ES, | | |
| | Golub TR. Multiclass cancer | | |
| | diagnosis using tumor gene | | |
| | expression signatures. Proc Natl | | |
| | Acad Sci USA. 2001 Dec 18; 98 | | |

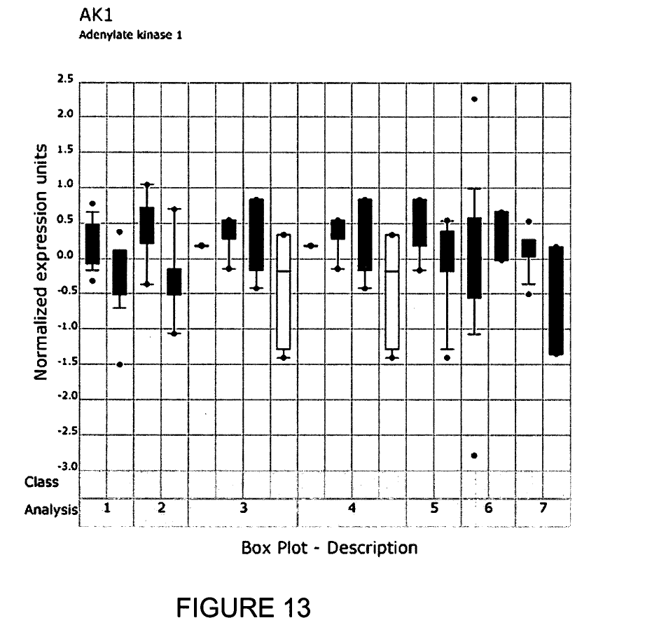

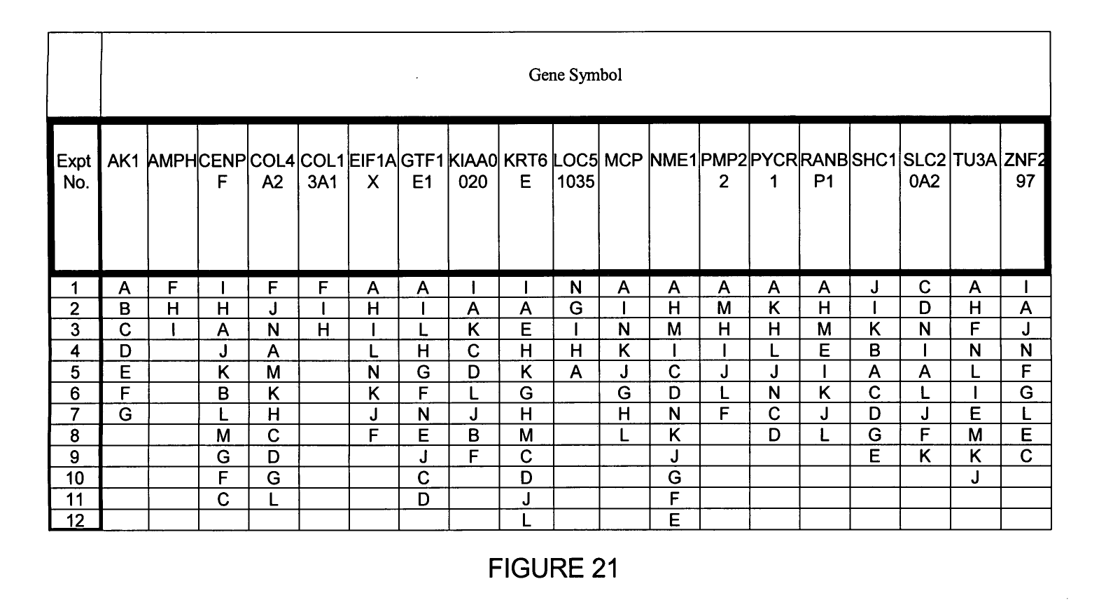

For the experiments for which the results are in the boxplots of FIGS. 2-20, the genes tested and the sample sets used are as noted in FIG. 21. The second row in the table of FIG. 21 notes the symbol of the gene being tested while the first column denotes the experiment number. The intersection between the gene symbol and the experiment number shows the sample set used for that experiment. The experiment number corresponds to the bottom row of the box plot for that gene. As an example, for the gene denoted by symbol AK1, the boxplot of which is in FIG. 13, experiment 1 used sample set A noted above. Since sample set A has two sample subsets, then there are two sub-columns for the first column in the box plot of FIG. 13. The first sub-column shows the expression level for the gene AK1 in normal tissue (as noted in the table above) while the second sub-column for this experiment is the expression level for the gene AK1 in adenocarcionoma tissue (again as noted above for sample set A).

As another example, experiment 7 for the gene PYCR1 used the sample set C with four subsample sets (see FIG. 2) which tested the expression level of PYCR1 in tissues at various Duke stages.

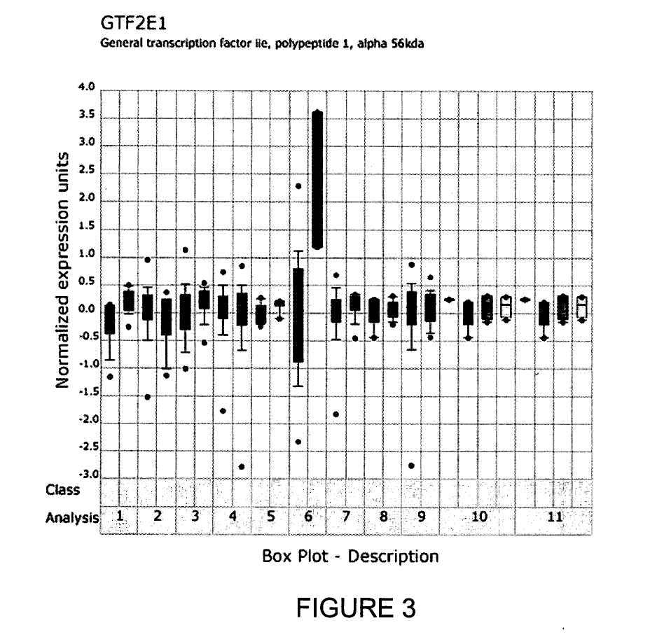

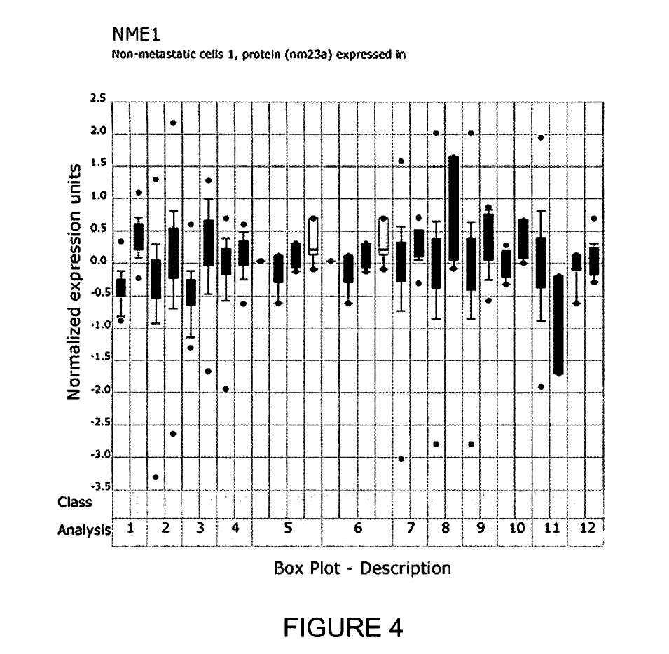

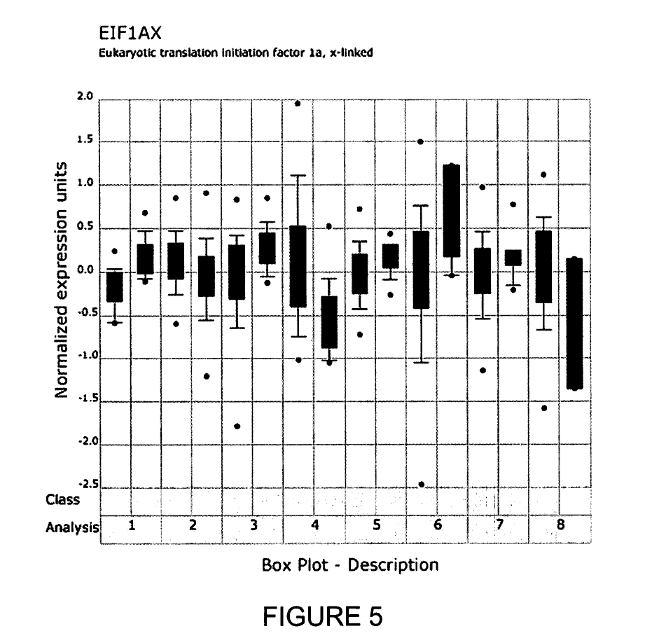

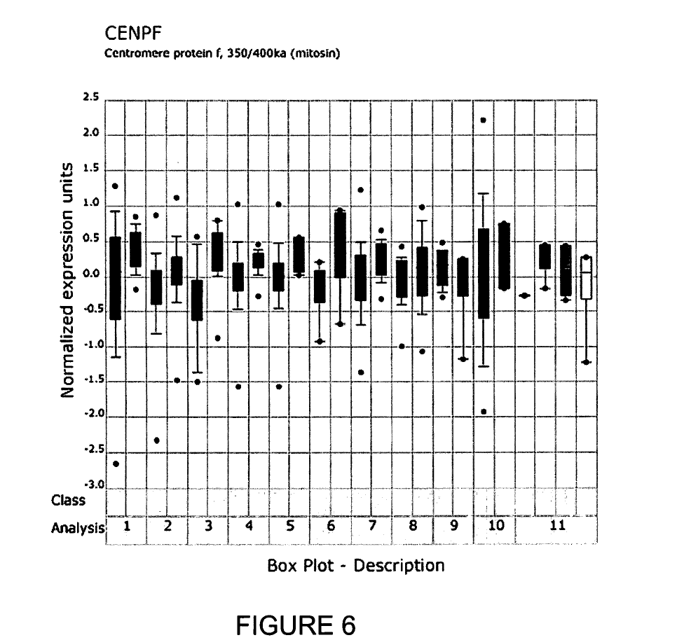

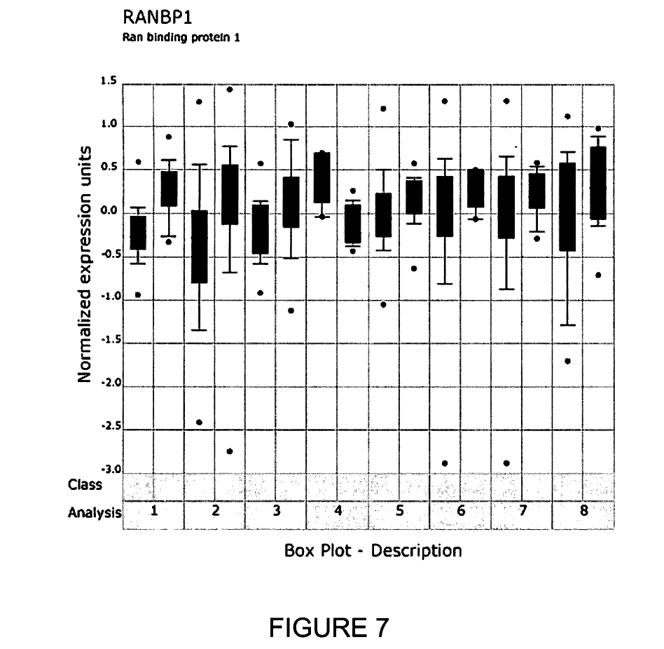

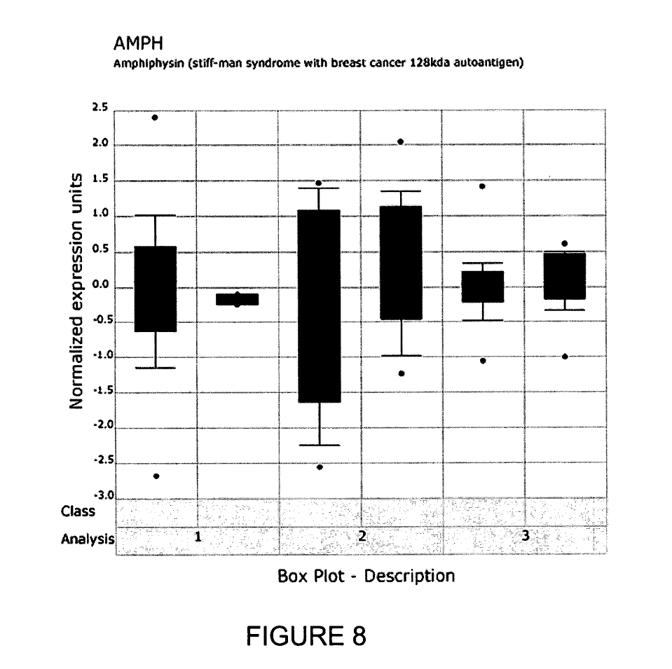

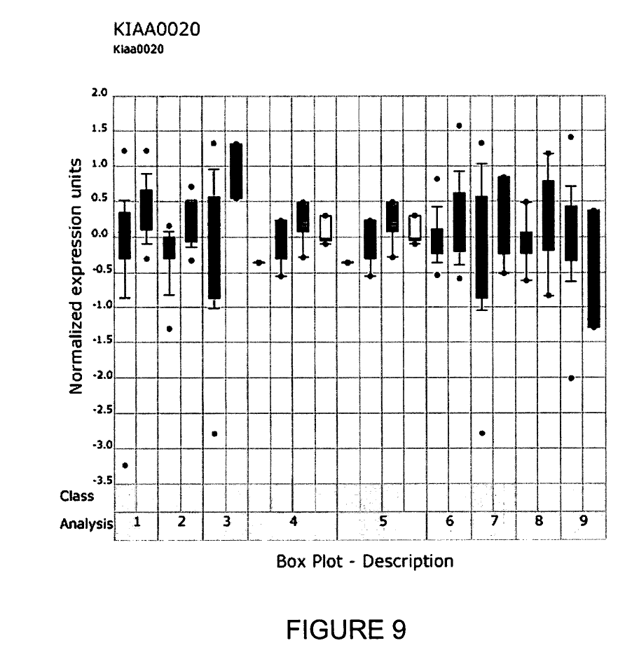

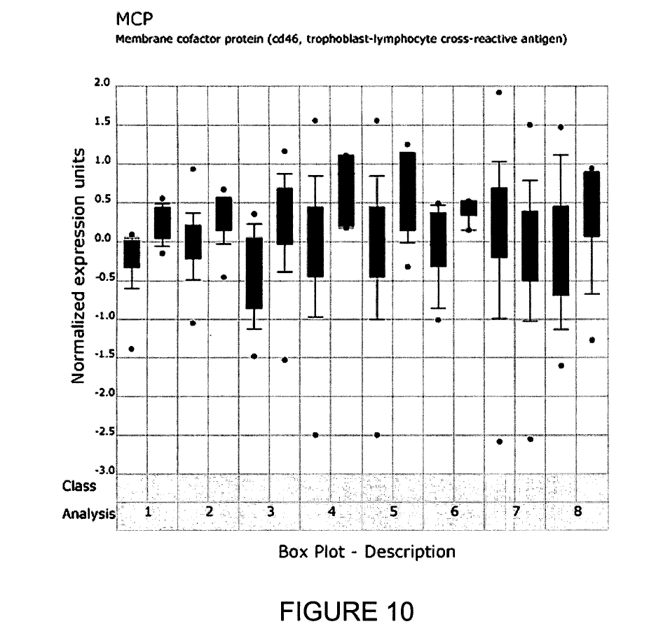

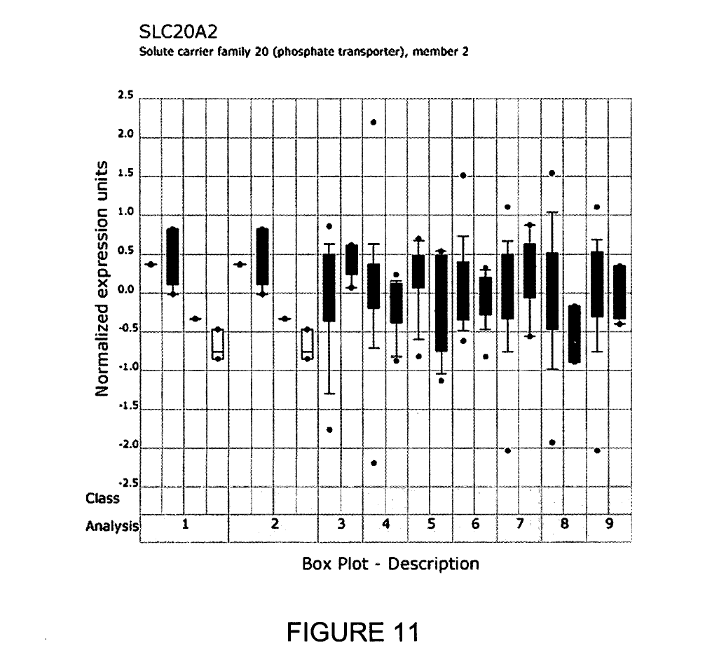

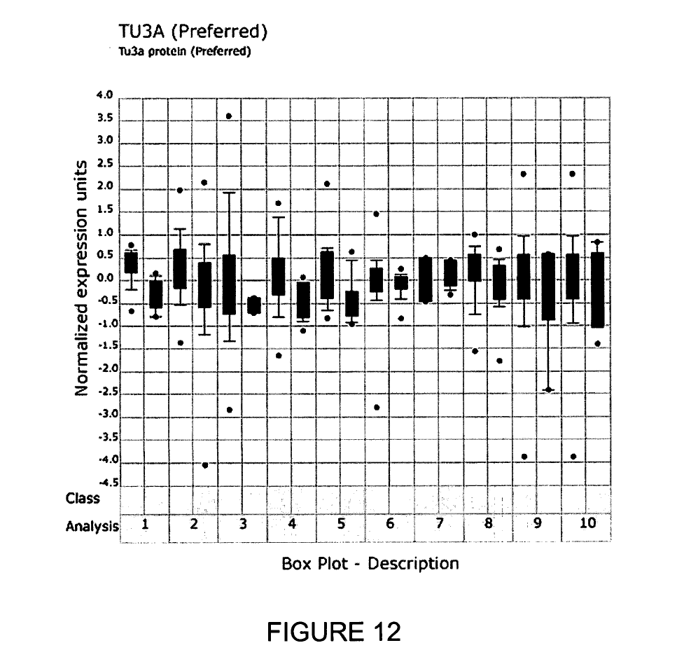

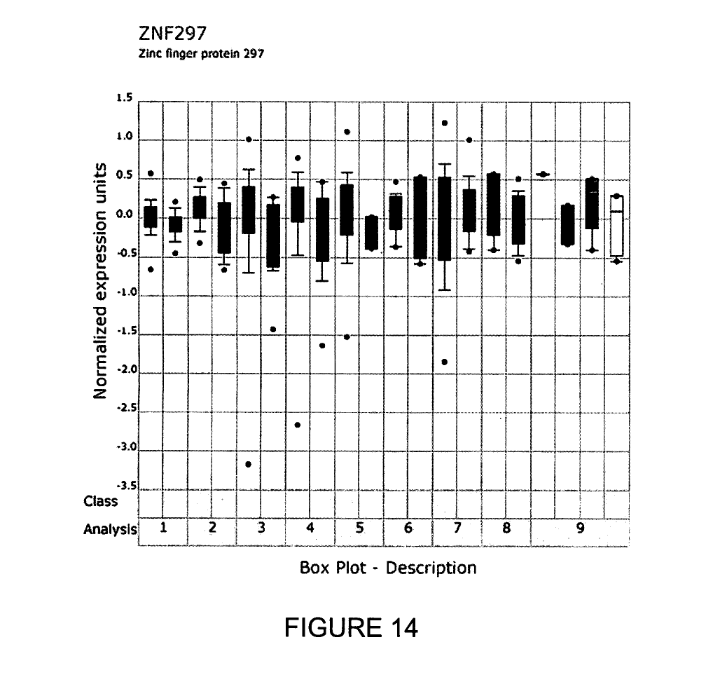

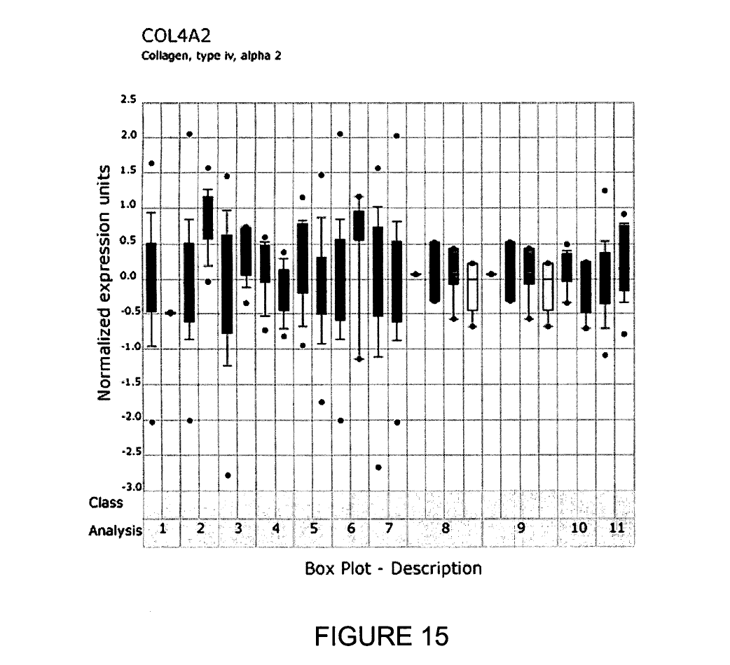

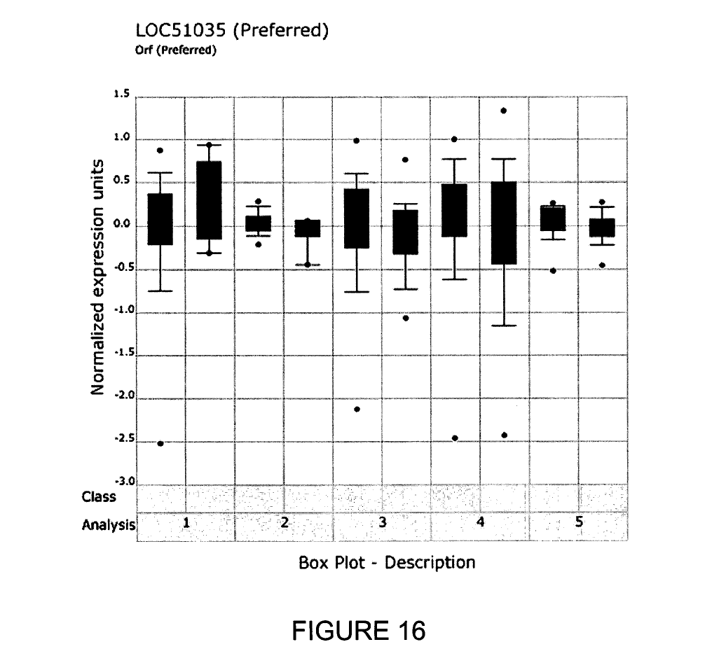

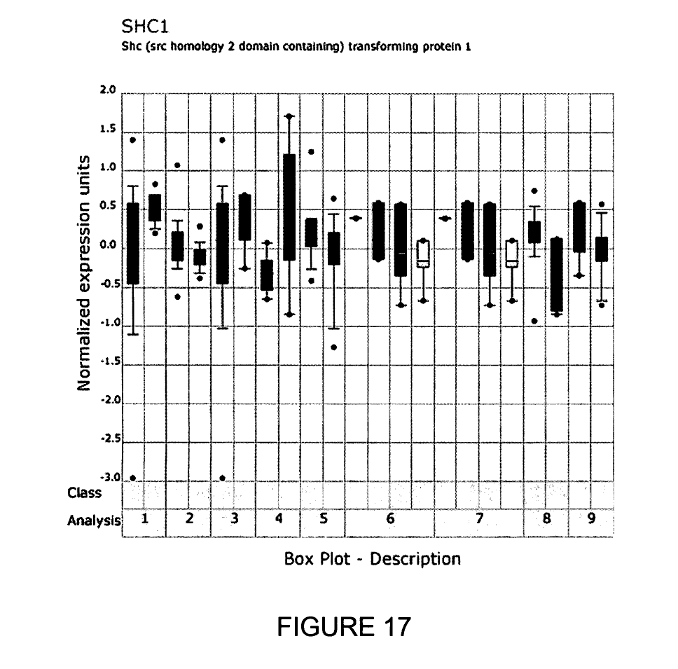

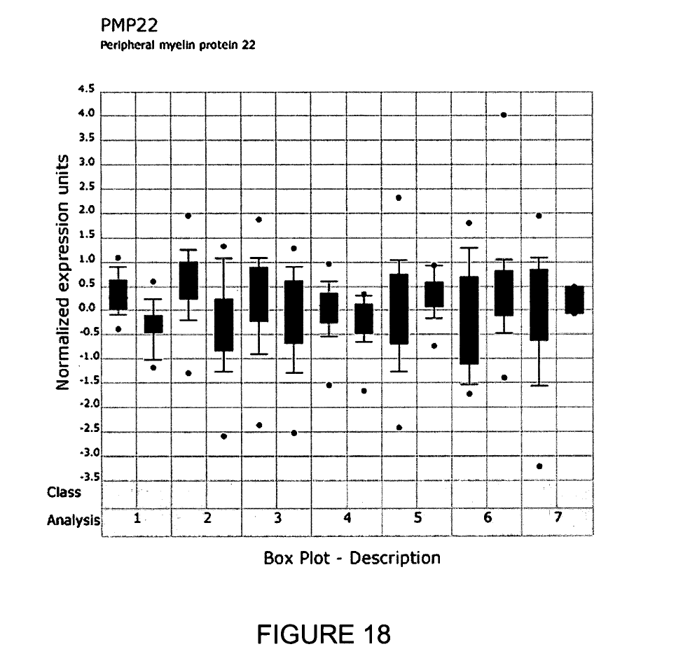

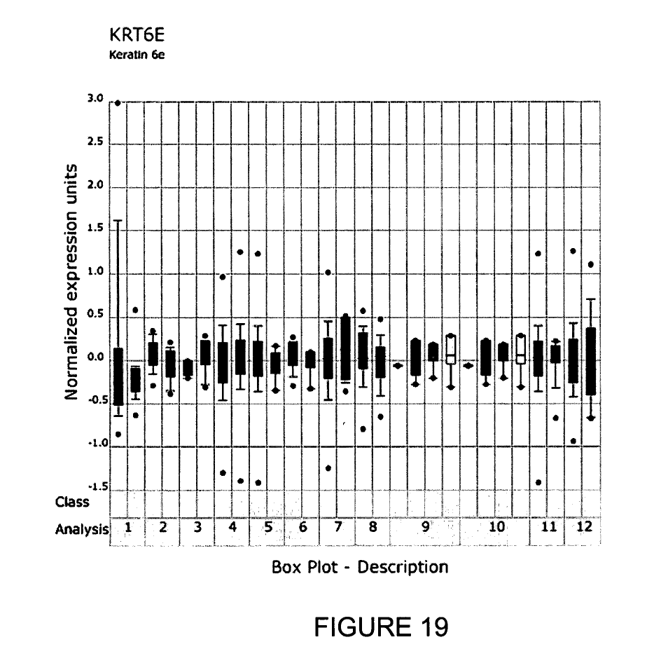

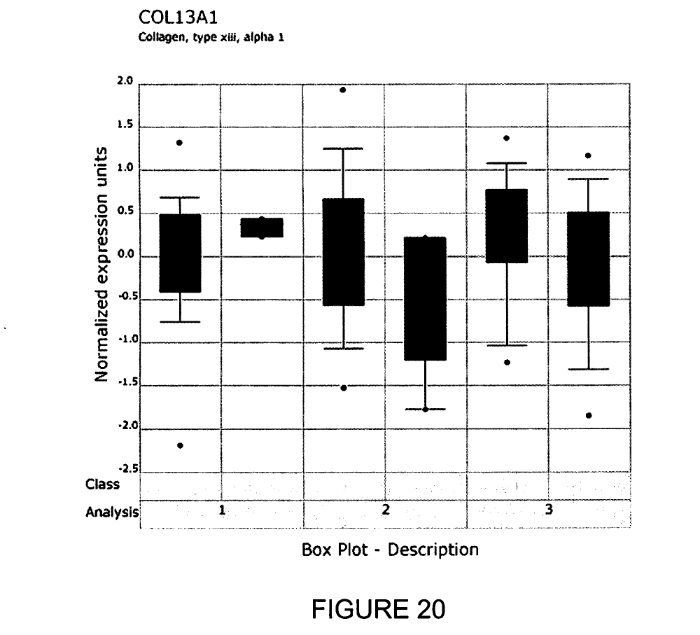

The correspondence between the test results in the figures and the genes being tested are as follows:

| Gene | Figure containing |
|---|---|
| Symbol | box plot results |
| PYCR1 | FIG. 2 |
| GTF2E1 | FIG. 3 |
| NME1 | FIG. 4 |
| EIF1AX | FIG. 5 |
| CENPF | FIG. 6 |
| AMPH | FIG. 7 |
| RANBP1 | FIG. 8 |
| KIAA0020 | FIG. 9 |
| MCP | FIG. 10 |
| SLC20A2 | FIG. 11 |
| TU3A | FIG. 12 |
| AK1 | FIG. 13 |
| ZNF297 | FIG. 14 |
| COL4A2 | FIG. 15 |
| LOC51035 | FIG. 16 |
| SHC | FIG. 17 |
| PMP22 | FIG. 18 |
| KRT6E | FIG. 19 |
| COL13A1 | FIG. 20 |

It should be noted that the underexpression or the overexpression of the above noted genes in cancerous tissue relative to their expression in normal tissue is readily evident in the box plots. Specifically, the experiments which used the samples sets A, B, M, and N compare the expression levels of specific genes in both cancerous and non-cancerous tissue in a side-by-side manner. For the genes which were not tested for sample sets A, B, M, and N, their expression levels for sample set F (normal tissue) may be compared with their expression levels for sample sets H and I (cancerous tissue). For the genes for which sample set E was used, the presence of p53 mutation indicates cancerous tissue, sample subset 2 for this sample set being cancerous tissue.

While it is preferable that the complete panel of 21 marker genes be used in the diagnosis of possible colon cancer, using a subset of the 21 marker genes will also yield useful results. Using a panel of anywhere from 1 to 21 marker genes out of the 21 marker genes on suspect colon tissue will still provide a useful indication as to whether cancerous colon tissue may be present or whether further and more involved tests are required.

A person understanding this invention may now conceive of alternative structures and embodiments or variations of the above all of which are intended to fall within the scope of the invention as defined in the claims that follow.

We claim:

1. A method for determining if colon cells are cancerous, the method comprising:
a) obtaining said colon cells;
b) determining if at least one specific gene is overexpressed or underexpressed in said colon cells compared to an expression of said at least one specific gene in normal colon cells;
c) determining that said colon cells are cancerous based on whether said at least one gene is overexpressed or underexpressed in said colon cells.

2. A method according to claim 1 wherein said colon cells are obtained by a core needle biopsy.

3. A method according to claim 1 wherein step b) comprises determining if a plurality of specific genes selected from a specific panel of marker genes are overexpressed in said colon cells.

4. A method according to claim 3 wherein step c) comprises determining that said colon cells are cancerous if said plurality of specific genes selected from said specific panel of marker genes are overexpressed in said colon cells.

5. A method according to claim 1 wherein step b) comprises determining if a plurality of specific genes selected from a selected panel of marker genes are underexpressed in said colon cells.

6. A method according to claim 5 wherein step c) comprises determining that said colon cells are cancerous if said plurality of specific genes selected from said specific panel of marker genes are underexpressed in said colon cells.

7. A method according to claim 1 wherein said at least one gene is selected from a group comprising:

| GeneBank | | |
|---|---|---|
| Accession | | |
| Number | Gene Name | Symbol |
| M77836 | Pyrroline-5-carboxylate | PYCR1 |
| | reductase 1 | |
| X63468 | General transcription | GTF2E1 |
| | factor IIE, polypeptide 1, | |
| | alpha 56 kDa | |
| H20426 | Transcribed locus, | NME1 |
| | strongly similar to | |
| | NP_937818.1 nucleoside- | |
| | diphosphate kinase 1 | |
| | isoform a [Homo sapiens] | |
| L18960 | Eukaryotic translation | EIF1AX |
| | initiation factor 1A, X- | |
| | linked | |
| U30872 | Centromere protein F, | CENPF |
| | 350/400ka (mitosin) | |
| X81438 | Amphiphysin (Stiff-Man | AMPH |
| | syndrome with breast | |
| | cancer 128 kDa | |
| | autoantigen) | |
| H67367 | RAN binding protein 1 | RANBP1 |
| D13645 | KIAA0020 | KIAA0020 |
| R33367 | Membrane cofactor | MCP |
| | protein (CD46, | |
| | trophoblast-lymphocyte | |
| | cross-reactive antigen) | |
| T94834 | similar to Homo sapiens | similar to |
| | acidic (leucine-rich) | ANP32B |
| | nuclear phosphoprotein | |
| | 32 family, member B. | |
| L20852 | Solute carrier family 20 | SLC20A2 |
| | (phosphate transporter), | |
| | member 2 | |
| R39540 | TU3A protein | TU3A |
| H86039 | Adenylate kinase 1 | AK1 |
| T78477 | Zinc finger protein 297 | ZNF297 |
| T84082 | ER Lumen Protein | KDELR1 |
| | Retaining Receptor 1 | |
| X05610 | Human mRNA for type IV | COL4A2 |
| | collagen alpha (2) chain | |
| R39130 | S27965 Hypothetical | LOC51035 |
| | protein | |
| T49397 | Src homology 2 domain | SHC |
| | contating transforming | |
| | protein 1 | |
| T94350 | Peripheral myelin protein | PMP22 |
| | 22 | |
| L42611 | Keratin 6 isoform K6e | KRT6E |
| | mRNA | |
| M33653 | Collagen, type XIII, | COL13A1 |
| | alpha 1 | |

8. A method according to claim 3 wherein said specific panel of marker genes comprises:

| GeneBank | | |
|---|---|---|
| Accession | | |
| Number | Gene Name | Symbol |
| M77836 | Pyrroline-5-carboxylate | PYCR1 |
| | reductase 1 | |
| X63468 | General transcription | GTF2E1 |
| | factor IIE, polypeptide 1, | |
| | alpha 56 kDa | |
| H20426 | Transcribed locus, | NME1 |
| | strongly similar to | |
| | NP_937818.1 nucleoside- | |
| | diphosphate kinase 1 | |
| | isoform a [Homo sapiens] | |
| L18960 | Eukaryotic translation | EIF1AX |
| | initiation factor 1A, X- | |
| | linked | |
| U30872 | Centromere protein F, | CENPF |
| | 350/400ka (mitosin) | |
| X81438 | Amphiphysin (Stiff-Man | AMPH |
| | syndrome with breast | |
| | cancer 128 kDa | |
| | autoantigen) | |
| H67367 | RAN binding protein 1 | RANBP1 |
| D13645 | KIAA0020 | KIAA0020 |
| R33367 | Membrane cofactor | MCP |
| | protein (CD46, | |
| | trophoblast-lymphocyte | |
| | cross-reactive antigen) | |
| T94834 | similar to Homo sapiens | similar to |
| | acidic (leucine-rich) | ANP32B |
| | nuclear phosphoprotein | |
| | 32 family, member B. | |

9. A method according to claim 5 wherein said specific panel of marker genes comprises:

| Gene Bank | | |
|---|---|---|
| Accession | | |
| No. | Gene Name | Symbol |
| L20852 | Solute carrier family 20 | SLC20A2 |
| | (phosphate transporter), | |
| | member 2 | |
| R39540 | TU3A protein | TU3A |
| H86039 | Adenylate kinase 1 | AK1 |
| T78477 | Zinc finger protein 297 | ZNF297 |
| T84082 | ER Lumen Protein | KDELR1 |
| | Retaining Receptor 1 | |
| X05610 | Human mRNA for type IV | COL4A2 |
| | collagen alpha (2) chain | |
| R39130 | S27965 Hypothetical | LOC51035 |
| | protein | |
| T49397 | Src homology 2 domain | SHC |
| | contating transforming | |
| | protein 1 | |
| T94350 | Peripheral myelin protein | PMP22 |
| | 22 | |
| L42611 | Keratin 6 isofom, K6e | KRT6E |
| | mRNA | |
| M33653 | Collagen, type XIII, | COL13A1 |
| | alpha 1 | |

10. Use of at least one marker gene for identifying cancerous colon tissue, an overexpression or underexpression of said at least one marker gene in colon tissue compared to an expression of said at least one marker gene in normal colon tissue being indicative of cancerous colon tissue.

11. A use according to claim 10 wherein an overexpression of said at least one marker gene is indicative of a presence of cancerous colon tissue, said at least one marker gene being selected from a group comprising:

| GeneBank | | |
|---|---|---|
| Accession | | |
| Number | Gene Name | Symbol |
| M77836 | Pyrroline-5-carboxylate | PYCR1 |
| | reductase 1 | |
| X63468 | General transcription | GTF2E1 |
| | factor IIE, polypeptide 1, | |
| | alpha 56 kDa | |
| H20426 | Transcribed locus, | NME1 |
| | strongly similar to | |
| | NP_937818.1 nucleoside- | |
| | diphosphate kinase 1 | |
| | isoform a [Homo sapiens] | |
| L18960 | Eukaryotic translation | EIF1AX |
| | initiation factor 1A, X- | |
| | linked | |
| U30872 | Centromere protein F, | CENPF |
| | 350/400ka (mitosin) | |
| X81438 | Amphiphysin (Stiff-Man | AMPH |
| | syndrome with breast | |
| | cancer 128 kDa | |
| | autoantigen) | |
| H67367 | RAN binding protein 1 | RANBP1 |
| D13645 | KIAA0020 | KIAA0020 |
| R33367 | Membrane cofactor | MCP |
| | protein (CD46, | |
| | trophoblast-lymphocyte | |
| | cross-reactive antigen) | |
| T94834 | similar to Homo sapiens | similar to |
| | acidic (leucine-rich) | ANP32B |
| | nuclear phosphoprotein | |
| | 32 family, member B. | |

12. A use according to claim 10 wherein an underexpression of said at least one marker gene is indicative of a presence of cancerous colon tissue, said at least one marker gene being selected from a group comprising:

| Gene Bank | | |
|---|---|---|
| Accession | | |
| No. | Gene Name | Symbol |
| L20852 | Solute carrier family 20 | SLC20A2 |
| | (phosphate transporter), | |
| | member 2 | |
| R39540 | TU3A protein | TU3A |
| H86039 | Adenylate kinase 1 | AK1 |
| T78477 | Zinc finger protein 297 | ZNF297 |
| T84082 | ER Lumen Protein | KDELR1 |
| | Retaining Receptor 1 | |
| X05610 | Human mRNA for type IV | COL4A2 |
| | collagen alpha (2) chain | |
| R39130 | S27965 Hypothetical | LOC51035 |
| | protein | |
| T49397 | Src homology 2 domain | SHC |
| | contating transforming | |
| | protein 1 | |
| T94350 | Peripheral myelin protein | PMP22 |
| | 22 | |
| L42611 | Keratin 6 isoform K6e | KRT6E |
| | mRNA | |
| M33653 | Collagen, type XIII, | COL13A1 |
| | alpha 1 | |

13. A method of diagnosing colon cancer, the method comprising:
a) obtaining colon tissue to be diagnosed;
b) determining if specific marker genes are overexpressed or underexpressed in said colon tissue to be diagnosed compared to non-cancerous colon tissue;
c) determining if said colon tissue to be diagnosed is cancerous based on an underexpression or overexpression of said specific marker genes.

14. A method according to claim 13 wherein said colon tissue is obtained by a core needle biopsy.

15. A method according to claim 13 wherein said specific marker genes are selected from a group comprising:

| GeneBank | | |
|---|---|---|
| Accession | | |
| Number | Gene Name | Symbol |
| M77836 | Pyrroline-5-carboxylate | PYCR1 |
| | reductase 1 | |
| X63468 | General transcription | GTF2E1 |
| | factor IIE, polypeptide 1, | |
| | alpha 56 kDa | |
| H20426 | Transcribed locus, | NME1 |
| | strongly similar to | |
| | NP_937818.1 nucleoside- | |
| | diphosphate kinase 1 | |
| | isoform a [Homo sapiens] | |
| L18960 | Eukaryotic translation | EIF1AX |
| | initiation factor 1A, X- | |
| | linked | |
| U30872 | Centromere protein F, | CENPF |
| | 350/400ka (mitosin) | |
| X81438 | Amphiphysin (Stiff-Man | AMPH |
| | syndrome with breast | |
| | cancer 128 kDa | |
| | autoantigen) | |
| H67367 | RAN binding protein 1 | RANBP1 |
| D13645 | KIAA0020 | KIAA0020 |
| R33367 | Membrane cofactor | MCP |
| | protein (CD46, | |
| | trophoblast-lymphocyte | |
| | cross-reactive antigen) | |
| T94834 | similar to Homo sapiens | similar to |
| | acidic (leucine-rich) | ANP32B |
| | nuclear phosphoprotein | |
| | 32 family, member B. | |
| L20852 | Solute carrier family 20 | SLC20A2 |
| | (phosphate transporter), | |
| | member 2 | |
| R39540 | TU3A protein | TU3A |
| H86039 | Adenylate kinase 1 | AK1 |
| T78477 | Zinc finger protein 297 | ZNF297 |
| T84082 | ER Lumen Protein | KDELR1 |
| | Retaining Receptor 1 | |
| X05610 | Human mRNA for type IV | COL4A2 |
| | collagen alpha (2) chain | |
| R39130 | S27965 Hypothetical | LOC51035 |
| | protein | |
| T49397 | Src homology 2 domain | SHC |
| | contating transforming | |
| | protein 1 | |
| T94350 | Peripheral myelin protein | PMP22 |
| | 22 | |
| L42611 | Keratin 6 isoform K6e | KRT6E |
| | mRNA | |
| M33653 | Collagen, type XIII, | COL13A1 |
| | alpha 1 | |

16. A method according to claim 15 wherein step b) comprises determining if a subset of said marker genes are overexpressed in said colon tissue to be diagnosed, the subset comprising:

| GeneBank | | |
|---|---|---|
| Accession | | |
| Number | Gene Name | Symbol |
| M77836 | Pyrroline-5-carboxylate | PYCR1 |
| | reductase 1 | |
| X63468 | General transcription | GTF2E1 |
| | factor IIE, polypeptide 1, | |
| | alpha 56 kDa | |
| H20426 | Transcribed locus, | NME1 |
| | strongly similar to | |
| | NP_937818.1 nucleoside- | |
| | diphosphate kinase 1 | |
| | isoform a [Homo sapiens] | |
| L18960 | Eukaryotic translation | EIF1AX |
| | initiation factor 1A, X- | |
| | linked | |
| U30872 | Centromere protein F, | CENPF |
| | 350/400ka (mitosin) | |
| X81438 | Amphiphysin (Stiff-Man | AMPH |
| | syndrome with breast | |
| | cancer 128 kDa | |
| | autoantigen) | |
| H67367 | RAN binding protein 1 | RANBP1 |
| D13645 | KIAA0020 | KIAA0020 |
| R33367 | Membrane cofactor | MCP |
| | protein (CD46, | |
| | trophoblast-lymphocyte | |
| | cross-reactive antigen) | |
| T94834 | similar to Homo sapiens | similar to |
| | acidic (leucine-rich) | ANP32B |
| | nuclear phosphoprotein | |
| | 32 family, member B. | |

17. A method according to claim 15 wherein step b) comprises determining if a subset of said marker genes are underexpressed in said colon tissue, the subset comprising:

| Gene Bank | | |
|---|---|---|
| Accession | | |
| No. | Gene Name | Symbol |
| L20852 | Solute carrier family 20 | SLC20A2 |
| | (phosphate transporter), | |
| | member 2 | |
| R39540 | TU3A protein | TU3A |
| H86039 | Adenylate kinase 1 | AK1 |
| T78477 | Zinc finger protein 297 | ZNF297 |
| T84082 | ER Lumen Protein | KDELR1 |
| | Retaining Receptor 1 | |
| X05610 | Human mRNA for type IV | COL4A2 |
| | collagen alpha (2) chain | |
| R39130 | S27965 Hypothetical | LOC51035 |
| | protein | |
| T49397 | Src homology 2 domain | SHC |
| | contating transforming | |
| | protein 1 | |
| T94350 | Peripheral myelin protein | PMP22 |
| | 22 | |
| L42611 | Keratin 6 isoform K6e | KRT6E |
| | mRNA | |
| M33653 | Collagen, type XIII, | COL13A1 |
| | alpha 1 | |

18. A method according to claim 1 wherein said colon cells are obtained from at least one stool sample.

19. A method according to claim 13 wherein said colon tissue are obtained from at least one stool sample.
